(19) United States
(12) Patent Application Publication
(10) Pub. No.: US 2025/0213504 A1
(43) Pub. Date: Jul. 3, 2025

(54) METHODS OF INTRAVENOUSLY ADMINISTERING SOTALOL HYDROCHLORIDE FOR THE TREATMENT OF PEDIATRIC PATIENTS

(71) Applicant: AltaThera Pharmaceuticals LLC, Chicago, IL (US)

(72) Inventors: Brandon Ira Kashfian, Chicago, IL (US); Jan Wald, Chicago, IL (US); Stanley Adam Strickberger, Chicago, IL (US); Alexander A. Vinks, Chicago, IL (US); Tomoyuki Mizuno, Chicago, IL (US)

(21) Appl. No.: 19/080,585

(22) Filed: Mar. 14, 2025

Related U.S. Application Data
(63) Continuation-in-part of application No. 19/009,774, filed on Jan. 3, 2025.
(63) Continuation-in-part of application No. 18/631,538, filed on Apr. 10, 2024.
(60) Provisional application No. 63/742,201, filed on Jan. 6, 2025.
(60) Provisional application No. 63/617,390, filed on Jan. 3, 2024.
(60) Provisional application No. 63/741,691, filed on Jan. 3, 2025.
(60) Provisional application No. 63/640,469, filed on Apr. 30, 2024.
(60) Provisional application No. 63/640,412, filed on Apr. 30, 2024.
(60) Provisional application No. 63/565,774, filed on Mar. 15, 2024.

(51) Int. Cl. A61K 31/145; A61K 9/00

(52) U.S. Cl. CPC A61K 31/145; A61K 9/0019

(57) ABSTRACT

Methods of administering sotalol hydrochloride in an amount effective for treating one or more cardiovascular disease or disorder in pediatric patients are described and include IV and/or oral doses based on age-specific creatinine clearance ranges.

### CROSS-REFERENCE TO RELATED APPLICATIONS

This application claims priority to and the benefit of the filing date of:
U.S. Provisional Application No. 63/742,201 filed Jan. 6, 2025;
U.S. patent application Ser. No. 19/009,774 filed Jan. 3, 2025;
   which claims priority to and the benefit of the filing date of U.S. Provisional Application No. 63/617,390, filed Jan. 3, 2024;
U.S. Provisional Application No. 63/741,691 filed Jan. 3, 2025;
U.S. Provisional Application No. 63/640,469 filed Apr. 30, 2024;
U.S. Provisional Application No. 63/640,412 filed Apr. 30, 2024;
U.S. patent application Ser. No. 18/631,538 filed Apr. 10, 2024; and
U.S. Provisional Application No. 63/565,774 filed Mar. 15, 2024; each of which is hereby incorporated by reference herein in its entirety.

### BACKGROUND OF THE INVENTION

### Field of the Invention

The present invention relates to the field of cardiovascular pharmaceutics, more particularly compositions and methods for the treatment of cardiovascular conditions, such as arrhythmias, with intravenous anti-arrhythmics, such as sotalol hydrochloride.

### Description of Related Art

Sotalol hydrochloride is a racemic mixture of d- and l-sotalol hydrochloride. Sotalol hydrochloride is a white, crystalline solid with a molecular weight of 308.8. It is hydrophilic, soluble in water, propylene glycol, and ethanol. Chemically, sotalol hydrochloride is a d,1-N-[4-[1-hydroxy-2-[(1-methylethyl)amino]ethyl]phenyl] methane-sulfonamide monohydrochloride. (HIGHLIGHTS OF PRESCRIBING INFORMATION: Sotalol hydrochloride injection for intravenous use, Revised 03/2020). The molecular formula is C12H2ON2O3S·HCl and is represented by the following structural formula:

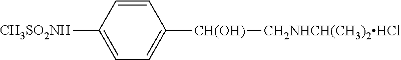

Intravenous sotalol is supplied as a sterile, clear solution in 10 ml vials, each vial containing 150 mg sotalol hydrochloride in acetate buffer. Sotalol hydrochloride is an antiarrhythmic drug with both beta adrenoreceptor blocking (Class II) and cardiac action potential duration prolongation (Class III) properties. The drug is hemodynamically well-tolerated and has a small risk of proarrhythmia. Both isomers have similar Class III activity, while the l-isomer is responsible for virtually all of the beta blocking activity. Sotalol does not have partial beta agonist or membrane stabilizing activity (Na channel inhibition). Sotalol does not undergo metabolism and is nearly 100% bioavailable when taken on an empty stomach. Sotalol hydrochloride does not bind to plasma proteins. The pharmacokinetics of d- and l-sotalol enantiomers are essentially identical. Excretion is predominantly via the kidney in the unchanged form and therefore lower doses are necessary in patients with renal impairment.

Sotalol is an effective antiarrhythmic agent but also can cause serious ventricular arrhythmias, primarily Torsade de Pointes (TdP) type ventricular tachycardia, a polymorphic ventricular tachycardia associated with QTc prolongation. QTc prolongation is directly related to the plasma concentration of sotalol. Conventionally, steady-state plasma levels of sotalol and maximum QTc prolongation are obtained in 3 days with oral administration (e.g., after 5-6 doses when administered twice daily). QTc changes are directly related to maximum serum concentration of the sotalol. To minimize the risk of sotalol caused arrhythmias, the product label for oral sotalol mandates that patients initiated or re-initiated on oral sotalol should be hospitalized for at least three days or until steady state drug levels are achieved, in a facility that can provide cardiac resuscitation and continuous electrocardiogramonitoring.

Hospitalization represents a high-cost burden in the United States. The three-day hospital stay is also burdensome to the patient, resulting in loss of productivity, time away from family, and an increased risk of hospital-acquired infections, as well as a diversion of hospital resources, staff, and patient care beds. The availability of IV sotalol offers an opportunity to improve on the sotalol IV protocol and decreases the length of hospital stay.

Sotalol is FDA approved for the maintenance of normal sinus rhythm in patients with history of highly symptomatic atrial fibrillation/flutter and for the treatment of documented life-threatening ventricular arrhythmias. Sotalol is currently approved in the US for oral administration (for example, under the brand name BETAPACE AF®, Bayer HealthCare Pharmaceuticals Inc.) and is approved for IV administration (AltaThera Pharmaceuticals LLC).

Sotalol may be implemented in the treatment and/or management of a variety of cardiovascular conditions, including for example, one or more of atrial flutter (AFL), atrial fibrillation, persistent atrial fibrillation, paroxysmal atrial fibrillation, ventricular fibrillation (VF), ventricular arrhythmias, ventricular tachycardia (VT), premature ventricular contractions (PVCs), paroxysmal atrial tachycardia, focal atrial tachycardia, supraventricular tachycardia (SVT), accessory pathway SVT, infantile SVT, post-operative SVT, paroxysmal supraventricular tachycardia (paroxysmal SVT), atrioventricular re-entrant tachycardia (AVRT), atrioventricular nodal re-entrant tachycardia (AVNRT), atrial tachycardia, junctional tachycardia, junctional ectopic tachycardia (JET), multifocal atrial tachycardia, atrial ectopic t tachycardia, hemodynamically stable or unstable ventricular tachycardia, congenital heart disease (CHD), heart failure, coronary artery disease, and pulmonary artery hypertension. (See, e.g., patent literature relating to IV sotalol, including U.S. Pat. Nos. 10,512,620, 10,799,138, 11,344,518, 11,583,216 11,610,660 and 11,696,902; and US Published Patent Application Nos. 2019/0307343, 2019/0380605, 2020/0085771, 2020/0093759, 2020/0253903, 2020/0338027, 2021/0076959, 2021/0283049, 2022/0142954, 2022/0241225, 2022/0339130, 2023/0075398, 2023/0172883, 2023/0187049, 2023/0225664, 2023/0225997, 2023/0248674, 2023/0255908, 2023/0270940, 2023/0293455, 2023/0372268, 2024/0156758 and 2024/0261242, which are each individually incorporated by reference herein in their entireties).

In pediatrics, sotalol has been shown to be useful for maintenance of normal sinus rhythm and for acute treatment (e.g., conversion) of tachycardia and arrhythmia (Borquez, A. et al., “Intravenous Sotalol in the Young”, JACC: Clinical Electrophysiology, 2020, Vol. 6, No. 4, 425-432; Valdes, S. et al., “Early Experience with Intravenous Sotalol in Children with and without Congenital Heart Disease”, Heart Rhythm, 2018, 15(12): 1862-1869; Celiker, A. et al., “Sotalol in treatment of pediatric cardiac arrhythmias”, Pediatrics International (2001) 43, 624-630; Pfammatter, J. et al., “Efficacy and Proarrhythmia of Oral Sotalol in Pediatric Patients”, JACC, Vol. 26, No. 4, 1995, 1002-1007; Marangnes, P. et al., “Usefulness of oral sotalol for the treatment of junctional ectopic tachycardia”, International Journal of Cardiology, 35 (1992) 165-167).

Junctional ectopic tachycardia (JET), a type of supraventricular tachycardia (SVT) which can occur in post-operative patients or as a congenital condition in infancy, has a particularly high mortality rate. There are two types of JET, idiopathic chronic junctional ectopic tachycardia (with structurally normal hearts) and transient postoperative junctional ectopic tachycardia (after repair of congenital heart disease). While the two types of JET have different causes, antiarrhythmic therapies, such as amiodarone, are often used in the treatment protocols (Batra, A. et al., “Junctional ectopic tachycardia: Current strategies for diagnosis and management”, Progress in Pediatric Cardiology, 35, 2013, 49-54).

For example, a typical treatment goal for JET is to decrease JET rate just low enough to pace over. The first line of treatment is management of symptoms, without administering antiarrhythmics. Then, the second line of treatment is the administration of a pharmacologic agent such as amiodarone or procainamide. Only as the third line of treatment is a first dose of IV sotalol administered (1 mg/kg intravenous sotalol over 1 hour), with a second dose considered in consultation with an EP specialist. (The Junctional Ectopic Tachycardia (JET) Care Guideline for Cardiovascular Intensive Care Unit (CVICU), 2019 CHOC Children's Hospital JET Guidelines.) The sotalol dosing is not adjusted for the patient's kidney function or creatinine clearance.

Oral administration protocols for sotalol in children for the treatment of ventricular arrhythmias, atrial fibrillation and/or atrial flutter also exist. (See Covis Pharma, Switzerland, BETAPACE®/BETAPACE AF, Full Prescribing Information, Revision 06/2021.) For example, one such regimen comprises administering 1.2 mg/kg three times a day (3.6 mg/kg total daily dose) (which is believed to be approximately equivalent to the initial 160 mg total daily dose for adults), followed by a maximum of 2.4 mg/kg three times a day (approximately equivalent to the 360 mg total daily dose for adults) with subsequent titration. The label indicates that titration should be guided by clinical response, heart rate, and QTc, with increased dosing being preferably conducted in-hospital. The regimen also indicates that at least 36 hours should be allowed between dose increments to attain steady-state plasma concentrations of sotalol in patients with age-adjusted normal renal function. The label further indicates that for children under 2 years of age, the dosage should be reduced by an age-dependent factor (e.g., 1 month, then 1.2 mg/kg×0.7 (age factor)=0.8 mg/kg). This regimen, however, is a one-size-fits-all approach for children with normal kidney function, in that all children of that age are administered the same weight-based dose and there is no adjustment for kidney function based on creatinine clearance.

The current IV sotalol hydrochloride label approved for use in the United States provides for suggested dosing for children. (HIGHLIGHTS OF PRESCRIBING INFORMATION: Sotalol hydrochloride injection for intravenous use, AltaThera Pharmaceuticals LLC, Revised 09/2023.) The regimen comprises administering a dose of IV sotalol hydrochloride to a pediatric patient in an amount of 30 mg/m² every 8 hours (which should give exposure similar to that with 160 mg daily in adults). A table is provided with suggested starting doses for children of age 3 days to greater than 12 years, with the children having normal renal function. For impaired renal function, the label provides general guidance for starting lower and titrating less frequently.

Thus, a need remains for effective protocols for pediatric sotalol hydrochloride treatment for acute treatment (such as restoring normal sinus rhythm or conversion to normal sinus rhythm), for maintenance of normal sinus rhythm, and for combinations thereof. In particular, effective sotalol dosing protocols are needed that provide customized dosing regimens for pediatric patients based on the patient's age, weight and kidney function (e.g., creatinine clearance).

### SUMMARY OF THE INVENTION

Described herein are methods of administering sotalol hydrochloride to a subject, such as a pediatric patient in need thereof, in an amount effective for treating a cardiovascular condition of the patient. Embodiments include methods of administering sotalol hydrochloride to a patient, such as a pediatric patient, comprising: administering an amount of sotalol, such as IV sotalol hydrochloride, to the patient according to an age-specific creatinine clearance level. Embodiments include methods comprising: obtaining a creatinine clearance of the patient; selecting an amount of IV sotalol hydrochloride to administer to the patient based on an age-specific creatinine clearance level; and administering the IV dose of sotalol hydrochloride to the patient.

Specific aspects of embodiments of the invention include:

Aspect 1, which is a method of administering sotalol hydrochloride treatment, comprising: determining age and creatinine clearance of a pediatric patient; selecting an amount of a single dose of sotalol hydrochloride to administer, wherein the amount of the single dose is an age-specific creatinine clearance based amount; and administering the single dose of sotalol hydrochloride to the pediatric patient for treatment of a cardiovascular condition; optionally administering one or more subsequent dose(s) of sotalol hydrochloride to the pediatric patient, which subsequent dose(s) comprise an amount of sotalol hydrochloride that is the same as, different from, or commensurate in effect with, the single dose. In embodiments, the dosing is based on TID dosing. In specific embodiments, the administering of the single dose and/or one or more of the subsequent doses can be selected according to Table 1:

TABLE 1
Sotalol IV Doses (mg/kg) in Children
According to Renal Function and Age

| | CrCl | |
|---|---|---|
| | (mL/min/ | Dose |
| Age | 1.73 m²) | (mg/kg) |
| 3 days | >33 | 0.33 |
| (0.1 mo.) | 22-33 | 0.26 |
| | 11-22 | 0.18 |
| | 4-11 | 0.12 |
| 6 days | >34 | 0.57 |
| (0.2 mo.) | 22-34 | 0.45 |
| | 11-22 | 0.32 |
| | 4-11 | 0.21 |
| 9 days | >34 | 0.72 |
| (0.3 mo.) | 23-34 | 0.57 |
| | 11-23 | 0.40 |
| | 4-11 | 0.27 |
| 12 days | >35 | 0.85 |
| (0.4 mo.) | 23-35 | 0.67 |
| | 12-23 | 0.48 |
| | 4-12 | 0.31 |
| 2 weeks | >36 | 0.89 |
| (0.5 mo.) | 24-36 | 0.70 |
| | 12-24 | 0.50 |
| | 4-12 | 0.33 |
| 3 weeks | >37 | 1.08 |
| | 25-37 | 0.85 |
| | 12-25 | 0.60 |
| | 4-12 | 0.40 |
| 1 month | >40 | 1.21 |
| | 26-40 | 0.96 |
| | 13-26 | 0.68 |
| | 4-13 | 0.45 |
| 3 months | >53 | 1.44 |
| | 35-53 | 1.14 |
| | 18-35 | 0.81 |
| | 6-18 | 0.53 |
| 6 months | >68 | 1.41 |
| | 45-68 | 1.12 |
| | 23-45 | 0.79 |
| | 8-23 | 0.52 |
| 12 months | >81 | 1.38 |
| | 54-81 | 1.09 |
| | 27-54 | 0.78 |
| | 9-27 | 0.51 |
| 2 years | >90 | 1.29 |
| | 60-90 | 1.02 |
| | 30-60 | 0.72 |
| | 10-30 | 0.48 |
| 6 years | >90 | 1.18 |
| | 60-90 | 0.93 |
| | 30-60 | 0.66 |
| | 10-30 | 0.43 |
| >12 years | >90 | 0.91 |
| | 60-90 | 0.72 |
| | 30-60 | 0.51 |
| | 10-30 | 0.34 |

CrCl = creatinine clearance

Aspect 2, which is the method of Aspect 1, wherein: the age of the patient is about 3 days, such as any patient less than 6 days old; and one or more of the doses is in an amount in the range of: (i) about 0.3-0.4 mg/kg and wherein the creatinine clearance of the patient is greater than 33 mL/min/1.73m²; or (ii) about 0.2-0.3 mg/kg and wherein the creatinine clearance of the patient is in the range of about 22 to 33 mL/min/1.73m²; or (iii) about 0.15-0.2 mg/kg and wherein the creatinine clearance of the patient is about 11 to 22 mL/min/1.73m²; or (iv) about 0.05-0.15 mg/kg and wherein the creatinine clearance of the patient is about 4 to 11 mL/min/1.73m².

Aspect 3 is the method of Aspect 1 or 2, wherein: the age of the patient is from 6 days to less than 9 days old; and one or more of the doses is in an amount ranging from: (i) about 0.5-0.6 mg/kg and wherein the creatinine clearance of the patient is greater than 34 mL/min/1.73m²; or (ii) about 0.4-0.5 mg/kg and wherein the creatinine clearance of the patient is about 22 to 34 mL/min/1.73m²; or (iii) about 0.3-0.4 mg/kg and wherein the creatinine clearance of the patient is about 11 to 22 mL/min/1.73m²; or (iv) about 0.1-0.3 mg/kg and wherein the creatinine clearance of the patient is about 4 to 11 mL/min/1.73m².

Aspect 4 is the method of any of Aspects 1-3, wherein: the age of the patient is in the range of 9 days to less than 12 days old; and one or more of the doses is in an amount ranging from: (i) about 0.6-0.8 mg/kg and wherein the creatinine clearance of the patient is greater than 34 mL/min/1.73m²; or (ii) about 0.5-0.6 mg/kg and wherein the creatinine clearance of the patient is about 23-34 mL/min/1.73m²; or (iii) about 0.3-0.5 mg/kg and wherein the creatinine clearance of the patient is about 11 to 23 mL/min/1.73m²; or (iv) about 0.2 to about 0.3 mg/kg and wherein the creatinine clearance of the patient is about 4 to 11 mL/min/1.73m².

Aspect 5 is the method of any of Aspects 1-4, wherein: the age of the patient is in the range of 12 days to less than 2 weeks; and one or more of the doses is in an amount ranging from: (i) about 0.7-0.9 mg/kg and wherein the creatinine clearance of the patient is greater than 35 mL/min/1.73m²; or (ii) about 0.5-0.7 mg/kg and wherein the creatinine clearance of the patient is about 23-35 mL/min/1.73m²; or (iii) about 0.4-0.5 mg/kg and wherein the creatinine clearance of the patient is about 12 to 23mL/min/1.73m²; or (iv) about 0.2-0.4 mg/kg and wherein the creatinine clearance of the patient is about 4-12 mL/min/1.73m².

Aspect 6 is the method of any of Aspects 1-5, wherein: the age of the patient is in the range of 2 weeks to less than 3 weeks old; and one or more of the doses is in an amount ranging from: (i) about 0.8-0.9 mg/kg and wherein the creatinine clearance of the patient is greater than 36 mL/min/1.73m²; or (ii) about 0.6-0.8 mg/kg and wherein the creatinine clearance of the patient is about 24 to 36 mL/min/1.73m²; or (iii) about 0.4-0.6 mg/kg and wherein the creatinine clearance of the patient is about 12 to 24 mL/min/1.73m²; or (iv) about 0.3-0.4 mg/kg and wherein the creatinine clearance of the patient is about 4 to 12 mL/min/1.73m².

Aspect 7 is the method of any of Aspects 1-6, wherein: the age of the patient is in the range of 3 weeks to less than 1 month old; and one or more of the doses is in an amount ranging from: (i) about 0.9-1.2 mg/kg and wherein the creatinine clearance of the patient is greater than 37 mL/min/1.73m²; or (ii) about 0.7-0.9 mg/kg and wherein the creatinine clearance of the patient is about 25 to 37 mL/min/1.73m²; or (iii) about 0.5-0.7 mg/kg and wherein the creatinine clearance of the patient is about 12 to 25 mL/min/1.73m²; or (iv) about 0.3-0.5 mg/kg and wherein the creatinine clearance of the patient is about 4 to 12 mL/min/1.73m².

Aspect 8 is the method of any of Aspects 1-7, wherein: the age of the patient is in the range of 1 month to less than 3 months; and one or more of the doses is in an amount ranging from: (i) about 1.1-1.3 mg/kg and wherein the creatinine clearance of the patient is greater than 40 mL/min/1.73m²; or (ii) about 0.8-1.1 mg/kg and wherein the creatinine clearance of the patient is about 26 to 40 mL/min/1.73m²; or (iii) about 0.5-0.8 mg/kg and wherein the creatinine clearance of the patient is about 13 to 26 mL/min/1.73m²; or (iv) about 0.3-0.5 mg/kg and wherein the creatinine clearance of the patient is about 4 to 13 mL/min/1.73m².

Aspect 9 is the method of any of Aspects 1-8, wherein: the age of the patient is in the range of 3 months to less than 6 months; and one or more of the doses is in an amount ranging from: (i) about 1.4-1.5 mg/kg and wherein the creatinine clearance of the patient is greater than 53 mL/min/1.73m²; or (ii) about 1.0-1.4 mg/kg and wherein the creatinine clearance of the patient is about 35 to 53 mL/min/1.73m²; or (iii) about 0.7-1.0 mg/kg and wherein the creatinine clearance of the patient is about 18 to 35 mL/min/1.73m²; or (iv) about 0.4-0.7 mg/kg and wherein the creatinine clearance of the patient is about 6 to 18 mL/min/1.73m².

Aspect 10 is the method of any of Aspects 1-9, wherein: the age of the patient is in the range of 6 months to less than 1 year; and one or more of the doses is in an amount ranging from: (i) about 1.35-1.45 mg/kg and wherein the creatinine clearance of the patient is greater than 68 mL/min/1.73m²; or (ii) about 0.9-1.35 mg/kg and wherein the creatinine clearance of the patient is about 45 to 68 mL/min/1.73m²; or (iii) about 0.6-0.9 mg/kg and wherein the creatinine clearance of the patient is about 23 to 45 mL/min/1.73m²; or (iv) about 0.3-0.6 mg/kg and wherein the creatinine clearance of the patient is about 8 to 23 mL/min/1.73m².

Aspect 11 is the method of any of Aspects 1-10, wherein: the age of the patient is in the range of 12 months to less than 2 years; and one or more of the doses is in an amount ranging from: (i) about 1.3-1.4 mg/kg and wherein the creatinine clearance of the patient is greater than 81 mL/min/1.73m²; or (ii) about 0.9-1.3 mg/kg and wherein the creatinine clearance of the patient is about 54 to 81 mL/min/1.73m²; or (iii) about 0.6-0.9 mg/kg and wherein the creatinine clearance of the patient is about 27 to 54 mL/min/1.73m²; or (iv) about 0.4-0.6 mg/kg and wherein the creatinine clearance of the patient is about 9 to 27 mL/min/1.73m².

Aspect 12 is the method of any of Aspects 1-11, wherein: the age of the patient is in the range of 2 years to less than 6 years; and one or more of the doses is in an amount ranging from: (i) about 1.27-1.35 mg/kg and wherein the creatinine clearance of the patient is greater than 90 mL/min/1.73m²; or (ii) about 0.9-1.27 mg/kg and wherein the creatinine clearance of the patient is about 60 to 90 mL/min/1.73m²; or (iii) about 0.6-0.9 mg/kg and wherein the creatinine clearance of the patient is about 30 to 60 mL/min/1.73m²; or (iv) about 0.3-0.6 mg/kg and wherein the creatinine clearance of the patient is about 10 to 30 mL/min/1.73m².

Aspect 13 is the method of any of Aspects 1-12, wherein: the age of the patient is in the range of 6-12 years; and one or more of the doses is in an amount ranging from: (i) about 1.0-1.3 mg/kg and wherein the creatinine clearance of the patient is greater than 90 mL/min/1.73m²; or (ii) about 0.7-1.0 mg/kg and wherein the creatinine clearance of the patient is about 60 to 90 mL/min/1.73m²; or (iii) about 0.5-0.7 mg/kg and wherein the creatinine clearance of the patient is about 30 to 59 mL/min/1.73m²; or (iv) about 0.2-0.5 mg/kg and wherein the creatinine clearance of the patient is about 10 to 30 mL/min/1.73m².

Aspect 14 is the method of any of Aspects 1-13, wherein: the age of the patient is greater than 12 years; and one or more of the doses is in an amount ranging from: (i) about 0.8-1.0 mg/kg and wherein the creatinine clearance of the patient is greater than 90 mL/min/1.73m²; or (ii) about 0.6-0.8 mg/kg and wherein the creatinine clearance of the patient is about 60 to 90 mL/min/1.73m²; or (iii) about 0.4-0.6 mg/kg and wherein the creatinine clearance of the patient is about 30 to 60 mL/min/1.73m²; or (iv) about 0.2-0.4 mg/kg and wherein the creatinine clearance of the patient is about 10 to 30 mL/min/1.73m².

Aspect 15 is the method of any of Aspects 1-14, wherein: in response to administering the single dose, the patient/subject is capable of achieving a peak serum, plasma or blood concentration of sotalol hydrochloride in the range of 90-110% of a Cₘₐₓ at steady-state that would be expected for that subject from oral dosing.

Aspect 16 is the method of any of Aspects 1-15, wherein: the subject/patient is currently in sinus rhythm, or is not currently in sinus rhythm, or has been or will be converted to sinus rhythm with sotalol or another antiarrhythmic and/or cardioversion such as electric cardioversion; and the subject/patient has a cardiovascular condition selected from atrial flutter (AFL), atrial fibrillation (AF), persistent atrial fibrillation, paroxysmal atrial fibrillation, paroxysmal or persistent atrial fibrillation or atrial flutter, a recent AF/AFL episode yet the patient is in sinus rhythm or will be cardioverted, ventricular fibrillation (VF) or ventricular arrhythmias, life-threatening recurrent VF or life-threatening recurrent hemodynamically unstable VT, focal atrial tachycardia, supraventricular tachycardia (SVT), atrioventricular re-entrant tachycardia (AVRT), atrioventricular nodal re-entrant tachycardia (AVNRT), atrial tachycardia, paroxysmal atrial tachycardia, junctional tachycardia, junctional ectopic tachycardia (JET), congenital heart disease (CHD), multifocal atrial tachycardia, atrial ectopic tachycardia, accessory pathway SVT, infantile SVT, post-operative SVT, paroxysmal SVT, treating Wolff-Parkinson-White syndrome, ventricular tachycardia (VT), premature ventricular contractions (PVCs), hemodynamically stable or unstable ventricular tachycardia, heart failure, coronary artery disease, and pulmonary artery hypertension, maintenance of normal sinus rhythm (for example, delay in time to recurrence of atrial fibrillation/atrial flutter (AFIB/AFL)) for example in patients with symptomatic AFIB/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function. In embodiments, sotalol can be administered to a patient who is receiving extracorporeal membrane oxygenation (ECMO).

Aspect 17 is the method of any of Aspects 1-16, comprising: determining a QT or QTc interval of the pediatric patient; and administering one or more subsequent dose(s) of sotalol hydrochloride; wherein one or more of the subsequent dose(s) is in an amount based on one or more QT or QTc interval of the pediatric patient.

Aspect 18 is the method of any of Aspects 1-17, wherein: the single dose is an IV dose; and one or more of the subsequent dose(s) are administered as IV dose(s); wherein the single IV dose and the one or more subsequent IV dose(s) are administered at an 8-hour interval.

Aspect 19 is the method of any of Aspects 1-18, wherein: the single dose is an IV dose or an oral dose; and the single IV dose and/or any one of more of the subsequent IV doses are administered as a bolus dose, or over a period of up to about 6 hours, such as over a period of up to about 0.1 hours, 0.2 hours, 0.25 hours, 0.3 hours, 0.4 hours, 0.5 hours, 0.75 hours, or 1, 2, 3, 4, 5, 6, 7, 8, 9, 10, 11, 12, 13, 14, 15, 17, 20, 25, 30, 35, 40, 45, 50, 55, 60, 65, 70, 75, 85, 90, 100, 115 or 120 minutes, or longer, such as over a period of up to about 1 hour, 1.25 hours, 1.5 hours, 1.75 hours, 2 hours, 2.25 hours, 2.5 hours, 2.75 hours, 3 hours, 3.5 hours, 4 hours, 4.5 hours, 5 hours, 5.5 hours or 6 hours. In preferred embodiments, the single IV dose and/or one or more of the subsequent IV doses is administered over a period of 30 min, 1 hour, or 0.5-1 hour.

### DETAILED DESCRIPTION OF VARIOUS EMBODIMENTS OF THE INVENTION

Reference will now be made in detail to various illustrative implementations. It is to be understood that the following discussion of implementations is not intended to be limiting.

AF is atrial fibrillation.

AFL is atrial flutter.

AF/AFL is atrial fibrillation and/or atrial flutter.

IV is intravenous.

JET refers to junctional ectopic tachycardia (JET), a type of supraventricular tachycardia (SVT) which can occur in post-operative patients or as a congenital condition in infancy, has a particularly high mortality rate. There are two types of JET, idiopathic chronic junctional ectopic tachycardia (with structurally normal hearts) and transient postoperative junctional ectopic tachycardia (after repair of congenital heart disease).

PO means “per os” and refers to an oral dosing regimen.

BID means “bis in die” and means twice a day.

TID means “ter in die” and means three times a day.

QD means “quaque die” and means once a day.

QID means “quater in die” and means four times a day.

Patient (or subject) refers to a human patient.

BP is blood pressure.

HR is heart rate.

Sotalol and sotalol hydrochloride (used interchangeably herein) refer to d,l-sotalol hydrochloride.

“Pediatric” patients/subjects as used herein refers to patients below the age of 21, such as patient who is up to 0.1 month, 0.1 months, 0.1-0.2 months, 0.2 months, 0.2-0.3 months, 0.3 months, 0.3-0.4 months, 0.4 months, 0.4-0.5 months, 0.5 months, 0.5 months to 3 weeks, 3 weeks, 3 weeks to 1 month, 1 month, 1-3 months, 3 months, 3-6 months, 6 months, 6-12 months, 12 months (1 year), 1-2 years, 2 years, 2-6 years, 6 years, 6-12 years, 12 years, or greater than 12 years old, such as 3 days, 6 days, 9 days, 12 days, 2 weeks, 3 weeks, 1 month, 3 months, 6 months, 1 year, 2 years, 6 years, or 12 years old or a pediatric patient over the age of 12 years, such as 13, 14, 15, 16, 17, 18, 19, 20 or 21 years old.

CrCl is creatinine clearance.

Age-specific creatinine clearance levels are shown in Table 2:

TABLE 2
Pediatric Age-specific Levels of Kidney Function (CrCl)

| | CrCl (mL/min/1.73 m²)-Kidney |
|---|---|
| Age | Function/Impairment |
| 3 days (0.1 month) | >33 (normal) |
| | 22-33 (mild) |
| | 11-22 (moderate) |
| | 4-11 (severe) |
| 6 days (0.2 months) | >34 (normal) |
| | 22-34 (mild) |
| | 11-22 (moderate) |
| | 4-11 (severe) |
| 9 days (0.3 months) | >34 (normal) |
| | 23-34 (mild) |
| | 11-23 (moderate) |
| | 4-11 (severe) |
| 12 days (0.4 months) | >35 (normal) |
| | 23-35 (mild) |
| | 12-23 (moderate) |
| | 4-12 (severe) |
| 2 weeks (0.5 months) | >36 (normal) |
| | 24-36 (mild) |
| | 12-24 (moderate) |
| | 4-12 (severe) |
| 3 weeks | >37 (normal) |
| | 25-37 (mild) |
| | 12-25 (moderate) |
| | 4-12 (severe) |
| 1 month | >40 (normal) |
| | 26-40 (mild) |
| | 13-26 (moderate) |
| | 4-13 (severe) |
| 3 months | ≥53 (normal) |
| | 35-53 (mild) |
| | 18-35 (moderate) |
| | 6-18 (severe) |
| 6 months | ≥68 (normal) |
| | 45-68 (mild) |
| | 23-45 (moderate) |
| | 8-23 (severe) |
| 12 months | >81 (normal) |
| | 54-81 (mild) |
| | 27-54 (moderate) |
| | 9-27 (severe) |
| 2 years | >90 (normal) |
| | 60-90 (mild) |
| | 30-60 (moderate) |
| | 10-30 (severe) |
| 6 years | >90 (normal) |
| | 60-90 (mild) |
| | 30-60 (moderate) |
| | 10-30 (severe) |
| >12 years | >90 (normal) |
| | 60-90 (mild) |
| | 30-60 (moderate) |
| | 10-30 (severe) |

CrCl = creatinine clearance

The terms “treat,” “treating,” and “treatment” refer to performing a method, such as administration of a drug, for the purpose of treating or ameliorating an injury, disease or condition, including methods resulting or not resulting in any indicia of success in the treatment or amelioration of an injury, disease, or condition, including any objective or subjective parameter such as abatement; remission; diminishing of symptoms or making the injury disease, or condition more tolerable to the subject; slowing in the rate of degeneration or decline; or improving a subject's well-being, such as physical or mental. The treatment or amelioration of symptoms can be based on objective or subjective parameters, including the results of a physical examination and/or other patient evaluation.

Cₘₐₓ ss is the maximal concentration obtained at steady state.

QT is the interval measured from the start of the Q wave or QRS complex, to the end of the T wave, where the Q wave corresponds to the beginning of ventricular depolarization and the T wave end corresponds to the end of ventricular repolarization.

QTc is the calculated interval that represents the QT interval corrected for heart rate and can be derived by mathematical correlation of the QT interval and the heart rate.

ΔQTc is the difference between a QTc measurement taken prior to the start of treatment and a QTc measured after the start of treatment.

The term “about” used herein in the context of quantitative measurements means the indicated amount ±10%. For example, “about 2 mg” can mean 1.8-2.2 mg.

Hospital refers to a medical facility staffed and equipped to provide continuous ECG monitoring and cardiac resuscitation to patients, if needed. Typically, the medical personnel are trained in the management of serious ventricular arrhythmias.

Reducing or shortening the length of a hospital stay refers to reducing/shortening the length of time a patient is admitted for oral sotalol initiation or escalation, for example at a hospital or other facility capable of providing cardiac resuscitation and continuous electrocardiogra monitoring. For example, a patient would typically require a 3-day (72 hour) stay in such hospital or facility to be initiated/escalated on oral sotalol, but can be released even on day 1 when following protocols according to the present invention.

### Formulations

Typically, IV sotalol is diluted for infusion. For example, IV sotalol can be diluted in saline, 5% dextrose in water (D5W), or Ringer's lactate. The dilution volume chosen is one that is convenient for administration and consistent with fluid restriction. A volumetric infusion pump can be used to administer the IV sotalol.

In an embodiment of the invention, the IV sotalol formulation comprises a sodium acetate buffer.

In an embodiment of the invention, sotalol hydrochloride is present in the IV formulation in an amount of up to about 30 mg/mL, such as up to about 5 mg/mL, 10 mg/mL, 15 mg/mL, 20 mg/mL, or 25 mg/mL. In an embodiment, sotalol hydrochloride is present in the IV formulation as a racemic mixture.

In an embodiment of the invention, the IV sotalol formulation is prepared with about 2.9 mg/mL glacial acetic acid in water and pH adjusted with sodium hydroxide to a pH of about 6-7.

### Indications

In embodiments, the patient is already in normal sinus rhythm prior to administering sotalol, and sotalol is indicated for the maintenance of normal sinus rhythm; and/or the patient is not in normal sinus rhythm prior to administering sotalol, and sotalol is administered for acute termination of arrhythmias, or sotalol is administered for acute termination of arrhythmias and maintenance of normal sinus rhythm.

In embodiments, the subject/patient is currently in sinus rhythm, or is not currently in sinus rhythm, or has been or will be converted to sinus rhythm with sotalol or another antiarrhythmic and/or cardioversion, such as electric cardioversion (e.g., DCCV or direct current cardioversion), and sotalol hydrochloride is administered for the treatment of one or more cardiovascular condition selected from atrial flutter (AFL), atrial fibrillation (AF), persistent atrial fibrillation, paroxysmal atrial fibrillation, treating paroxysmal or persistent atrial fibrillation or atrial flutter, such as with patients who have experienced a recent AF/AFL episode who are in sinus rhythm or who will be cardioverted, treating ventricular fibrillation (VF) or ventricular arrhythmias, treating life-threatening recurrent VF or life-threatening recurrent hemodynamically unstable VT, treating focal atrial tachycardia, supraventricular tachycardia (SVT), atrioventricular re-entrant tachycardia (AVRT), atrioventricular nodal re-entrant tachycardia (AVNRT), atrial tachycardia, paroxysmal atrial tachycardia, junctional tachycardia, junctional ectopic tachycardia (JET), congenital heart disease (CHD), multifocal atrial tachycardia, atrial ectopic tachycardia, accessory pathway SVT, infantile SVT, post-operative SVT, paroxysmal SVT, treating Wolff- Parkinson-White syndrome, ventricular tachycardia (VT), premature ventricular contractions (PVCs), hemodynamically stable or unstable ventricular tachycardia, heart failure, coronary artery disease, and pulmonary artery hypertension, maintenance of normal sinus rhythm (for example, delay in time to recurrence of atrial fibrillation/atrial flutter (AF/AFL)) for example in patients with symptomatic AF/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function. In embodiments, sotalol can be administered to a pediatric patient who is receiving extracorporeal membrane oxygenation (ECMO).

Typically, other antiarrhythmic therapy is withdrawn prior to starting sotalol.

### Sotalol Hydrochloride Dosing

In embodiments of the invention, one or more IV dose is delivered by way of an IV bolus or infusion over a period of up to about 6 hours, such as over a period of up to about 0.1 hours, 0.2 hours, 0.25 hours, 0.3 hours, 0.4 hours, 0.5, 0.75 hours, or 1, 2, 3, 4, 5, 6, 7, 8, 9, 10, 11, 12, 13, 14, 15, 17, 20, 25, 30, 35, 40, 45, 50, 55, 60, 65, 70, 75, 85, 90, 100, 115 or 120 minutes, or longer, such as over a period of up to about 1 hour, 1.25 hours, 1.5 hours, 1.75 hours, 2 hours, 2.25 hours, 2.5 hours, 2.75 hours, 3 hours, 3.5 hours, 4 hours, 4.5 hours, 5 hours, 5.5 hours or 6 hours. In preferred embodiments, the single IV dose and/or one or more of the subsequent IV doses is administered over a period of 30 min, 1 hour, or 0.5-1 hour. An IV dose can be administered while the patient/subject is admitted to a hospital or other facility capable of providing cardiac resuscitation and continuous electrocardiogramonitoring or is administered to the patient after the patient is discharged from the hospital or monitoring facility, with optional electrocardiogramonitoring outside of the facility.

In embodiments, the IV dosing may be dosed by body weight (in mg/kg) or by body surface area (BSA), expressed as mg/m². In embodiments, the body surface area is calculated according to Equation 1.

$$BSA ( {m}^{2} ) = \sqrt{\frac{[ height ( cm ) \times weight ( kg )}{3600}} \qquad Equation ( 1 )$$

In embodiments, the IV dose of sotalol hydrochloride is administered according to body weight (mg/kg). In embodiments, the one or more IV dose and/or the total daily dose (optionally split into two or more IVs or a combination of oral and IV doses) is administered according to body weight and is administered in an amount of up to about 5 mg/kg, such as up to about 0.05 mg/kg, 0.1 mg/kg, 0.15 mg/kg, 0.2 mg/kg, 0.25 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, or 4.5 mg/kg.

In embodiments, the IV dose is administered according to body surface area (mg/m²). In embodiments, the one or more IV dose and/or the total daily dose (optionally split into two or more IVs or a combination of oral and IV doses) of sotalol hydrochloride is administered according to body surface area and is administered in an amount of up to about 100 mg/m2, such as up to about 1 mg/m², 1.5 mg/m², 2 mg/m², 3 mg/m², 3 mg/m², 4 mg/m², 4.5 mg/m², 5 mg/m², 6 mg/m², 7 mg/m², 8 mg/m², 9 mg/m², 10 mg/m², 12 mg/m², 15 mg/m², 18 mg/m², 20 mg/m², 21 mg/m², 22 mg/m², 24 mg/m², 25 mg/m², 27 mg/m², 28 mg/m², 30 mg/m², 32 mg/m², 33 mg/m², 35 mg/m², 36 mg/m², 38 mg/m², 39 mg/m², 40 mg/m², 42 mg/m², 45 mg/m², 48 mg/m², 50 mg/m², 52 mg/m², 55 mg/m², 58 mg/m², 60 mg/m², 62 mg/m², 64 mg/m², 65 mg/m², 70 mg/m², 75 mg/m², 80 mg/m², 85 mg/m², 90 mg/m², or 95 mg/m², or any range within any of these values as endpoints. The potency of intravenous sotalol is thought to be similar in children, so a dose of 30 mg/m² every 8 hours is expected to give exposure similar to that with 160 mg daily in adults.

In embodiments, sotalol hydrochloride can be administered with a daily amount of up to about 30 mg/m², 60 mg/m², 90 mg/m², 120 mg/m², 150 mg/m², 180 mg/m², or 210 mg/m², for example. In embodiments the daily amount is administered in equal doses Q.D., B.I.D., T.I.D., or Q.I.D. In other embodiments, the daily amount is administered in unequal doses.

In embodiments, one or more sotalol hydrochloride dose(s) is/are administered orally, such as administering an IV dose followed by oral dosing. In embodiments, the oral dose and/or the total daily dose (optionally split into two or more oral doses or a combination of oral and IV doses) of sotalol hydrochloride is administered according to body weight (mg/kg), such as in an amount of up to about 5 mg/kg, such as up to about 0.05 mg/kg, 0.1 mg/kg, 0.15 mg/kg, 0.2 mg/kg, 0.25 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, or 4.5 mg/kg. Alternatively, or in addition, the oral dose and/or the total daily dose (optionally split into two or more oral doses or a combination of oral and IV doses) is administered according to body surface area (mg/m²), such as in an amount of up to about 100 mg/m², such as up to about 1 mg/m², 1.5 mg/m², 2 mg/m², 3 mg/m², 3 mg/m², 4 mg/m², 4.5 mg/m², 5 mg/m², 6 mg/m², 7 mg/m², 8 mg/m², 9 mg/m², 10 mg/m², 12 mg/m², 15 mg/m², 18 mg/m², 20 mg/m², 21 mg/m², 22 mg/m², 24 mg/m², 25 mg/m², 27 mg/m², 28 mg/m², 30 mg/m², 33 mg/m², 35 mg/m², 36 mg/m², 39 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², or 95 mg/m². The potency of intravenous sotalol is thought to be similar in children, so a dose of 30 mg/m² every 8 hours is expected to give exposure similar to that with 160 mg daily in adults.

In embodiments, multiple doses of sotalol hydrochloride are administered to the patient. The multiple doses are administered orally, intravenously, or as a combination thereof. For example, a first dose may be administered by way of an IV dose, then a subsequent dose may be administered intravenously and an additional subsequent dose may be administered orally. Further, for example, an IV dose may be followed by one or more subsequent IV doses and/or one or more subsequent oral doses. In embodiments, the first IV dose can comprise the same amount of sotalol hydrochloride and be administered over the same duration as one or more subsequent IV dose, or the first IV dose can comprise the same amount of sotalol hydrochloride as one or more subsequent IV dose and be administered over a different duration of time, or the first IV dose can comprise a different amount of sotalol hydrochloride as one or more subsequent IV dose and be administered over a different duration of time as the subsequent IV dose, such as a longer or shorter period of time.

In embodiments, a subsequent dose (IV or oral dose) is administered immediately or up to about 48 hours after the start or conclusion of the IV dose, such as up to about 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.25 hours, 5.5 hours, 5.75 hours, 6 hours, 6.25 hours, 6.5 hours, 6.75 hours, 7 hours, 7.25 hours, 7.5 hours, 7.75 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 32 hours, 40 hours or 48 hours after administration of the IV dose.

In embodiments, the first subsequent dose (IV or oral dose) is administered about 1 hour to about 48 hours after the start or conclusion of the administration of the first dose, such as about 2 hours to about 11 hours, about 3 hours to about 10 hours, about 4 hours to about 9 hours, about 5 hours to about 8 hours, or about 6 hours to about 7 hours after administration of the first dose, or 12-24 hours or 24-48 hours after.

In embodiments, the subject/patient is administered a first dose (IV dose or oral dose) and at least one subsequent dose (IV dose and/or oral dose) prior to being released from the hospital or other facility that was providing the monitoring, or the patient is administered all or at least one subsequent dose after being discharged. In embodiments, after being released, the patient/subject can still be monitored in a similar way, such as by using a portable/wearable ECG monitoring system.

In embodiments, one or more of the subsequent dose(s) is administered according to body weight (mg/kg). In embodiments, the total daily dose and/or one or more of the individual dose(s) (IV or oral dose), such as an initial dose or a subsequent dose, is administered according to body weight and is administered in an amount of up to about 5 mg/kg, such as up to about 0.05 mg/kg, 0.1 mg/kg, 0.15 mg/kg, 0.2 mg/kg, 0.25 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, or 4.5 mg/kg.

In embodiments, the subsequent dose (IV or oral dose) is administered according to body surface area (mg/m²) and optionally can have an upper limit, such as a maximum dose of about 80 mg, 120 mg, or 160 mg. In embodiments, the total daily dose and/or one or more of the dose(s) is administered according to body surface area and is administered in an amount of up to about 100 mg/m², such as up to about 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², or 95 mg/m². Some doses can be administered according to body weight and other doses according to BSA.

In embodiments, more than one dose amount is given to a particular patient (e.g., a patient receives one or more dose at 2 mg/kg, a subsequent higher dose of 3 mg/kg or lower dose of 1 mg/kg, or any combination thereof).

In embodiments, one or more of the doses is selected based on a patient's change in QT interval or QTc (for example, a patient that experiences an increase in QTc of less than 20% after receiving the first dose may receive a higher oral dose than a patient that experiences an increase in QTc of 20% or greater after receiving the first dose). In embodiments, the dose is changed after administration of a previous subsequent dose due to a change in the patient's QTc (for example, a patient given a first subsequent dose of 40 mg/m2 experiences an increase in QTc of 20% or greater, and the next subsequent dose can be lowered to 30 mg/m2).

In embodiments, additional doses are given after the first dose. In embodiments, the additional doses are administered at intervals of about 4 hours, about 8 hours, about 12 hours, about 16 hours, about 20 hours, about 24 hours, about 32 hours, about 40 hours, or about 48 hours.

In embodiments, one or more of the first dose amount and/or subsequent dose amount, delay between administration of the first dose and any subsequent dose(s), and/or interval between subsequent doses is/are selected based on one or more of the patient's age, sex, creatinine clearance, weight and/or change in QT interval or QTc, such as the patient's age, sex, weight and creatinine clearance, or based on the patient's age, weight and creatinine clearance and optionally is/are selected based on an age-specific creatinine clearance range.

In embodiments, the first dose and/or subsequent dose(s) are given in amounts and at time intervals such that the patient achieves or is capable of achieving C_{max ss} within about 48 hours, such as within about 24 hours, within about 8 hours, or even within about 1 or 2 hours, of the start or completion of the administration of the first dose. In embodiments, C_{max ss} is achieved before administration of a subsequent dose. In embodiments, C_{max ss} is achieved after administration of a subsequent dose. In embodiments, in response to administering an IV dose, the patient/subject is capable of achieving a peak serum, plasma or blood concentration of sotalol hydrochloride in the range of 90-110% of a Cₘₐₓ at steady-state that would be expected for that subject from chronic oral dosing.

In embodiments, a first dose is administered in an amount such that the patient experiences a Cₘₐₓ that is at least about 80% of the C_{max ss} for the patient's specific dosing protocol within about 1-3 hours of initiation of the dose. In embodiments, following the initial IV dose, such as before a subsequent dose (IV or oral dose), the patient experiences or is capable of experiencing a Cₘₐₓ that is at least about 85% of the C_{max ss}, such as about 87%, 90%, 92%, 95%, 97% or 99% of the C_{max ss} for the patient's specific dosing protocol.

### Creatinine Clearance

In embodiments, one or more of the doses is/are selected based on a patient's creatinine clearance and according to age-specific creatinine clearance ranges. Creatinine clearance can be calculated using the Schwartz Equation or Updated Schwartz Equation, or the Cockcroft-Gault formula as appropriate.

The Cockcroft-Gault formulas for creatinine clearance (CrCl) are:

$$CrCl ( male ) = ( ( 140 - age ) \times weight in kg ) / ( serum creatinine \times 72 )$$

$$CrCl ( female ) = CrCl ( male ) \times 0.85$$

The Schwartz Equation for creatinine clearance (CrCl) in pediatric patients is:

$$CrCl = k \times height in cm / serum creatinine in mg / dL$$

Values for “k” are given in Table 3.

TABLE 3
Values for Schwartz Equation

| Age | k value |
|---|---|
| Low birth weight babies, <1 year old | 0.33 |
| Term (average gestational age) babies, <1 year old | 0.45 |
| Children (1-12) and adolescent girls (13-17 years) | 0.55 |
| Adolescent boys (13-17 years) | 0.70 |

The Updated Schwartz Equation for creatinine clearance in pediatric patients is:

$$CrCl = 0.413 \times height in cm / serum creatinine in mg / dL$$

In some cases, a patient/subject with a lower creatinine clearance may receive a lower dose of sotalol hydrochloride than a patient with a higher creatinine clearance, or in some cases a patient/subject with a higher creatinine clearance may receive a lower dose than a patient with a lower creatinine clearance. In some cases, for example, the amount of sotalol hydrochloride, whether by IV or an oral dose, appropriate to administer to a subject with a CrCl of 10-30 mL/min or 30-60 mL/min can be higher than is appropriate for a subject with a CrCl of >90 mL/min. Further, for example, a subject/patient of younger age may receive a higher dose of sotalol hydrochloride than an older subject/patient, such as a 6 month old or 1 year old with a normal creatinine clearance who would receive about 1.38 mg/kg, while a 2-6 year old with a normal creatinine clearance would receive about 1.29 mg/kg, while a 6 year old or a 12 year old with normal creatinine clearance would receive 1.18 mg/kg, and a >12 year old subject/patient with a normal creatinine clearance would receive about 0.91 mg/kg.

The following examples are intended to be illustrative and should not be interpreted to limit the claimed subject matter.

### Age-Specific Creatinine Clearance Based Dosing

In embodiments, the sotalol hydrochloride dosing amount is selected from an amount appropriate for the pediatric patient based on an age-specific creatinine clearance level and is based on a patient's age, weight and/or creatinine clearance. Further exemplary dose (mg/kg) ranges, according to embodiments of the invention, are shown in Table 4.

TABLE 4
Exemplary Dosing (mg/kg) in Children
According to Renal Function and Age

| | CrCl | |
|---|---|---|
| | (mL/min/ | Dose |
| Age | 1.73 m²) | (mg/kg) |
| 3 days | >33 | 0.3-0.4 |
| (0.1 mo.) | 22-33 | 0.2-0.3 |
| | 11-22 | 0.15-0.2 |
| | 4-11 | 0.05-0.15 |
| 6 days | >34 | 0.5-0.6 |
| (0.2 mo.) | 22-34 | 0.4-0.5 |
| | 11-22 | 0.3-0.4 |
| | 4-11 | 0.1-0.3 |
| 9 days | >34 | 0.6-0.8 |
| (0.3 mo.) | 23-34 | 0.5-0.6 |
| | 11-23 | 0.3-0.5 |
| | 4-11 | 0.2-0.3 |
| 12 days | >35 | 0.7-0.9 |
| (0.4 mo.) | 23-35 | 0.5-0.7 |
| | 12-23 | 0.4-0.5 |
| | 4-12 | 0.2-0.4 |
| 2 weeks | >36 | 0.8-0.9 |
| (0.5 mo.) | 24-36 | 0.6-0.8 |
| | 12-24 | 0.4-0.6 |
| | 4-12 | 0.3-0.4 |
| 3 weeks | >37 | 0.9-1.2 |
| | 25-37 | 0.7-0.9 |
| | 12-25 | 0.5-0.7 |
| | 4-12 | 0.3-0.5 |
| 1 month | >40 | 1.1-1.3 |
| | 26-40 | 0.8-1.1 |
| | 13-26 | 0.5-0.8 |
| | 4-13 | 0.3-0.5 |
| 3 months | >53 | 1.4-1.5 |
| | 35-53 | 1.0-1.4 |
| | 18-35 | 0.7-1.0 |
| | 6-18 | 0.4-0.7 |
| 6 months | >68 | 1.35-1.45 |
| | 45-68 | 0.9-1.35 |
| | 23-45 | 0.6-0.9 |
| | 8-23 | 0.3-0.6 |
| 12 | >81 | 1.3-1.4 |
| months | 54-81 | 0.9-1.3 |
| | 27-54 | 0.6-0.9 |
| | 9-27 | 0.4-0.6 |
| 2 years | >90 | 1.27-1.35 |
| | 60-90 | 0.9-1.27 |
| | 30-60 | 0.6-0.9 |
| | 10-30 | 0.3-0.6 |
| 6 years | >90 | 1.0-1.3 |
| | 60-90 | 0.7-1.0 |
| | 30-60 | 0.5-0.7 |
| | 10-30 | 0.2-0.5 |
| >12 years | >90 | 0.8-1.0 |
| | 60-90 | 0.6-0.8 |
| | 30-60 | 0.4-0.6 |
| | 10-30 | 0.2-0.4 |

CrCl = creatinine clearance

Further examples of sotalol hydrochloride dosing, e.g., IV or oral dosing, can be chosen from the values shown in Table 1 (above and reproduced below).

TABLE 1
Sotalol IV Doses (mg/kg) in Children
According to Renal Function and Age

| | CrCl | |
|---|---|---|
| | (mL/min/ | Dose |
| Age | 1.73 m²) | (mg/kg) |
| 3 days | >33 | 0.33 |
| (0.1 mo.) | 22-33 | 0.26 |
| | 11-22 | 0.18 |
| | 4-11 | 0.12 |
| 6 days | >34 | 0.57 |
| (0.2 mo.) | 22-34 | 0.45 |
| | 11-22 | 0.32 |
| | 4-11 | 0.21 |
| 9 days | >34 | 0.72 |
| (0.3 mo.) | 23-34 | 0.57 |
| | 11-23 | 0.40 |
| | 4-11 | 0.27 |
| 12 days | >35 | 0.85 |
| (0.4 mo.) | 23-35 | 0.67 |
| | 12-23 | 0.48 |
| | 4-12 | 0.31 |
| 2 weeks | >36 | 0.89 |
| (0.5 mo.) | 24-36 | 0.70 |
| | 12-24 | 0.50 |
| | 4-12 | 0.33 |
| 3 weeks | >37 | 1.08 |
| | 25-37 | 0.85 |
| | 12-25 | 0.60 |
| | 4-12 | 0.40 |
| 1 month | >40 | 1.21 |
| | 26-40 | 0.96 |
| | 13-26 | 0.68 |
| | 4-13 | 0.45 |
| 3 months | >53 | 1.44 |
| | 35-53 | 1.14 |
| | 18-35 | 0.81 |
| | 6-18 | 0.53 |
| 6 months | >68 | 1.41 |
| | 45-68 | 1.12 |
| | 23-45 | 0.79 |
| | 8-23 | 0.52 |
| 12 months | >81 | 1.38 |
| | 54-81 | 1.09 |
| | 27-54 | 0.78 |
| | 9-27 | 0.51 |
| 2 years | >90 | 1.29 |
| | 60-90 | 1.02 |
| | 30-60 | 0.72 |
| | 10-30 | 0.48 |
| 6 years | >90 | 1.18 |
| | 60-90 | 0.93 |
| | 30-60 | 0.66 |
| | 10-30 | 0.43 |
| >12 years | >90 | 0.91 |
| | 60-90 | 0.72 |
| | 30-60 | 0.51 |
| | 10-30 | 0.34 |

CrCl = creatinine clearance

In embodiments, sotalol hydrochloride dosing can be expressed in mg/m². Exemplary dose ranges (mg/m²), according to embodiments of the invention, are shown in Table 5.

TABLE 5
Exemplary Dosing (mg/m²) in Children
According to Renal Function and Age

| | | CrCl | |
|---|---|---|---|
| | | (mL/min/ | Dose |
| Age | | 1.73 m²) | (mg/m²) |
| 0.1 | mo. | >33 | 9-12 |
| | | 22-33 | 6-9 |
| | | 11-22 | 4.5-6 |
| | | 4-11 | 1.5-4.5 |
| 0.2 | mo. | >34 | 15-18 |
| | | 22-34 | 12-15 |
| | | 11-22 | 9-12 |
| | | 4-11 | 3-9 |
| 0.3 | mo. | >34 | 18-24 |
| | | 23-34 | 15-18 |
| | | 11-23 | 9-15 |
| | | 4-11 | 6-9 |
| 0.4 | mo. | >35 | 21-27 |
| | | 23-35 | 15-21 |
| | | 12-23 | 12-15 |
| | | 4-12 | 6-12 |
| 0.5 | mo. | >36 | 24-27 |
| | | 24-36 | 18-27 |
| | | 12-24 | 12-18 |
| | | 4-12 | 9-18 |
| 3 | weeks | >37 | 27-36 |
| | | 25-37 | 21-27 |
| | | 12-25 | 15-21 |
| | | 4-12 | 9-15 |
| 1 | mo. | >40 | 33-39 |
| | | 26-39 | 24-33 |
| | | 13-26 | 15-24 |
| | | 4-13 | 9-15 |
| 3 | | >53 | 39-45 |
| | mos. | 35-53 | 30-39 |
| | | 18-35 | 21-30 |
| | | 6-17 | 12-21 |
| 6 | mos. | >68 | 36-45 |
| | | 45-68 | 27-36 |
| | | 23-45 | 18-27 |
| | | 8-23 | 9-18 |
| 12 | mos. | >81 | 36-45 |
| | | 54-81 | 27-36 |
| | | 27-54 | 18-27 |
| | | 9-27 | 12-18 |
| 2 | years | >90 | 33-42 |
| | | 60-90 | 27-33 |
| | | 30-60 | 18-27 |
| | | 10-30 | 9-18 |
| 6 | years | >90 | 30-39 |
| | | 60-90 | 21-30 |
| | | 30-60 | 15-21 |
| | | 10-30 | 6-15 |
| 12 | years | >90 | 24-30 |
| | | 60-90 | 18-24 |
| | | 30-60 | 12-18 |
| | | 10-30 | 6-12 |

CrCl = creatinine clearance

Further examples of BSA-based sotalol hydrochloride dosing are shown in Table 6.

TABLE 6
Sotalol IV Doses (mg/m²) in Children
According to Renal Function and Age

| | | CrCl | |
|---|---|---|---|
| | | (mL/min/ | Dose |
| Age | | 1.73 m²) | (mg/m²) |
| 0.1 | mo. | >33 | 9.9 |
| | | 22-33 | 7.8 |
| | | 11-22 | 5.4 |
| | | 4-11 | 3.6 |
| 0.2 | mo. | >34 | 17.1 |
| | | 22-34 | 13.5 |
| | | 11-22 | 9.6 |
| | | 4-11 | 6.3 |
| 0.3 | mo. | >34 | 21.6 |
| | | 23-34 | 17.1 |
| | | 11-23 | 12 |
| | | 4-11 | 8.1 |
| 0.4 | mo. | >35 | 25.5 |
| | | 23-35 | 20.1 |
| | | 12-23 | 14.4 |
| | | 4-12 | 9.3 |
| 0.5 | mo. | >36 | 26.7 |
| | | 24-36 | 21 |
| | | 12-24 | 15 |
| | | 4-12 | 9.9 |
| 3 | weeks | >37 | 32.4 |
| | | 25-37 | 25.5 |
| | | 12-25 | 18 |
| | | 4-12 | 12 |
| 1 | mo. | >40 | 36.3 |
| | | 26-40 | 28.8 |
| | | 13-26 | 20.4 |
| | | 4-13 | 13.5 |
| 3 | mos. | >53 | 43.2 |
| | | 35-53 | 34.2 |
| | | 18-35 | 24.3 |
| | | 6-18 | 15.9 |
| 6 | mos. | >68 | 42.3 |
| | | 45-68 | 33.6 |
| | | 23-45 | 23.7 |
| | | 8-23 | 15.6 |
| 12 | mos. | >81 | 41.4 |
| | | 54-81 | 32.7 |
| | | 27-54 | 23.4 |
| | | 9-27 | 15.3 |
| 2 | years | >90 | 38.7 |
| | | 60-90 | 30.6 |
| | | 30-60 | 21.6 |
| | | 10-30 | 14.4 |
| 6 | years | >90 | 35.4 |
| | | 60-90 | 27.9 |
| | | 30-60 | 19.8 |
| | | 10-30 | 12.9 |
| 12 | years | >90 | 27.3 |
| | | 60-90 | 21.6 |
| | | 30-60 | 15.3 |
| | | 10-30 | 10.2 |

### EXAMPLE 1—1 Year Old With AFL and Age-Specific CrCl in Normal Range

An example sotalol treatment protocol for a pediatric patient experiencing AFL, who is or is not currently in normal sinus rhythm is described herein. The male patient, age 1 year, would be admitted to initiate treatment in a hospital or other facility capable of providing cardiac resuscitation and continuous electrocardiogramonitoring. The patient's creatinine clearance is determined to be normal based on age and is determined to be 90 mL/min/1.73m². The patient is connected to an electrocardiogramd an initial QTc is determined to be less than 450 ms. A physician determines the patient should receive sotalol therapy at an amount that provides exposure similar to a dose of 160 mg daily in adults. Based on the patient's creatinine clearance being in the normal range for the patient's age, treatment is initiated with a first IV dose of sotalol hydrochloride in an amount in the range of 1.3-1.4 mg/kg, such as 1.38 mg/kg over a period of about 10 minutes. The patient's QTc is measured following the IV dose administration, along with heart rate and blood pressure. The patient's QTc does not exceed 500 ms, and the AQTc is less than 20%.

Once it is determined that the patient/subject is capable of tolerating the sotalol by way of the first IV dose, one or more subsequent IV or oral dose(s) can be administered to the subject at an 8-hour interval (for example, 8 hours from the start of the first IV dose), whether still admitted to the hospital or after discharge. In embodiments, the subsequent oral doses are administered in an amount of about 30 mg/m² sotalol hydrochloride every 8 hours, and the subsequent IV doses are administered in an amount in the range of 1.3-1.4 mg/kg (which can be the same or a different amount than the first IV dose) over a period of about 1 hour at an 8-hour interval. The patient's QTc is monitored following administration of the subsequent oral or IV dose. The patient's QTc still does not exceed 500 ms and the patient's AQTc is less than 20%.

### EXAMPLE 2—2 Month Old With AFL and Kidney Function With Mild Impairment

An example sotalol treatment protocol for a pediatric patient experiencing AFL, who is or is not currently in normal sinus rhythm is described herein. The female patient, age 2 months, would be admitted to initiate treatment in a hospital or other facility capable of providing cardiac resuscitation and continuous electrocardiogramonitoring. The patient's creatinine clearance is determined to be 35 mL/min/1.73m², which indicates a kidney function of mild impairment according to the age-specific creatinine clearance levels for children aged 1 month up to 3 months. The patient is connected to an electrocardiogramd an initial QTc is determined to be less than 450 ms. A physician determines the patient should receive sotalol therapy at an amount that provides exposure similar to that with 160 mg daily in adults. Treatment is initiated with an IV dose of sotalol hydrochloride in an amount in the range of 0.8-1.1 mg/kg, such as 0.96 mg/kg over a period of about 1 hour. The patient's QTc interval is measured every 15 minutes during the IV dose administration, along with heart rate and blood pressure. The patient's QTc does not exceed 500 ms during any of these measurements, and the AQTc is less than 20%.

If it is determined that the patient/subject is capable of tolerating the sotalol by way of the first IV dose, one or more subsequent IV doses in an amount in the range of 0.8-1.1 mg/kg (which can be the same or a different amount than the first IV dose) over a period of about 1 hour can be given to the patient to provide exposure similar to that with 160 mg daily in adults, such as at an 8-hour interval. The subsequent IV doses can be administered in a facility capable of providing cardiac resuscitation and continuous electrocardiogramonitoring if the patient's QTc is monitored and still does not exceed 500 ms and the patient's AQTc is less than 20%.

### EXAMPLE 3—2 Week Old With JET and Age-Specific CrCl in Normal Range

An example sotalol treatment protocol for a pediatric patient experiencing JET is described herein. The male patient, age 2 weeks, would be admitted to initiate treatment in a hospital or other facility capable of providing cardiac resuscitation and continuous electrocardiogramonitoring. The patient's creatinine clearance is determined to be normal based on age. The patient is connected to an electrocardiogramd an initial QTc is determined to be less than 450 ms. A physician determines the patient should be initiated on an acute sotalol dosing regimen. Treatment is initiated with an IV dose of sotalol hydrochloride in an amount of 0.89 mg/kg over a period of about 30 minutes. The patient's QTc is measured every 15 minutes during the IV dose administration, along with heart rate and blood pressure. The patient's QTc does not exceed 500 ms during any of these measurements, and the AQTc is less than 20%.

During or after administration of the IV dose, a physician would determine if cardioversion has occurred and the patient should be transitioned to a sotalol dosing regimen. A subsequent dose of 0.89 mg/kg would be administered via IV infusion administered over a period of up to 5 hours, such as over a period of about 30 min. A second subsequent dose of 0.89 mg/kg would be administered by IV over a period of 1-5 hours, such as over a period of about 30 min. or about 1 hour, after initiation of the first subsequent dose. Any additional subsequent doses would be administered according to a T.I.D. dosing protocol.

### EXAMPLE 4—6 Month Old With JET and Severe Kidney Function Impairment

An example sotalol treatment protocol for a pediatric patient experiencing JET is described herein. The female patient, age 6 months, is 2 days post-op from surgery related to correction of a congenital heart defect. Treatment would be initiated in a hospital or other facility capable of providing cardiac resuscitation and continuous electrocardiogramonitoring. The patient's creatinine clearance indicates severe kidney function impairment. The patient is connected to an electrocardiogramd an initial QTc is determined to be less than 450 ms. A physician determines the patient should be initiated on an acute sotalol dosing regimen. Treatment is initiated with an IV dose of sotalol hydrochloride in an amount in the range of 0.3-0.6 mg/kg, such as 0.52 mg/kg, over a period of about 1 hour. The patient's QTc interval is measured every 15 minutes during the IV dose administration, along with heart rate and blood pressure. The patient's QTc does not exceed 500 ms during any of these measurements, and the ΔQTc is less than 20%.

During or after administration of the IV dose, a physician would determine if cardioversion has occurred and the patient should receive one or more subsequent sotalol doses. A first subsequent dose of 0.52 mg/kg would be administered orally or by IV within 8 hours of the initiation of the IV dose and additional subsequent doses of 0.52 mg/kg would be administered every 8 hours from the first subsequent dose, such as over a period of about 1 hour.

### EXAMPLE 5—3 Day Old With AFL, Not Currently in Normal Sinus Rhythm

An example sotalol treatment protocol for a pediatric patient experiencing AFL, who is not currently in normal sinus rhythm is described herein. The male patient, age 0.1 month, would be admitted to initiate treatment in a hospital or other facility capable of providing cardiac resuscitation and continuous electrocardiogramonitoring. The patient's creatinine clearance is determined to be 15 mL/min/1.73 m², which indicates moderate impairment of kidney function according to the age-specific creatinine clearance ranges. The patient is connected to an electrocardiogramd an initial QTc is determined to be less than 450 ms. A physician determines the patient should be initiated on an IV sotalol dosing regimen. Treatment is initiated with an IV dose of sotalol hydrochloride in an amount of about 0.15-0.2 mg/kg, such as 0.18 mg/kg over a period of about 30 minutes. The patient's QTc is measured during the IV dose administration, along with heart rate and blood pressure. The patient's QTc does not exceed 500 ms and the AQTc is less than 20%.

Once it is determined that the patient/subject is capable of tolerating the sotalol by way of the first IV dose, a subsequent IV dose in the range of 0.15-0.2 mg/kg (which can be the same or a different amount than the first IV dose) can be administered over a period of up to 5 hours, such as about 10 min., 30 min., 1 hour or 5 hours, to the subject about 8 hours after initiation of the first IV dose. The patient's QTc is monitored following administration of the subsequent IV dose. The patient's QTc still does not exceed 500 ms and the patient's ΔQTc is less than 20%. Additional subsequent IV doses in an amount in the range of 0.15-2.0 mg/kg, such as 0.18 mg/kg would be administered to the patient every 8 hours, such as over a period of up to 5 hours as indicated prior.

### EXAMPLE 6-6 Year Old With AFL and Mild Renal Impairment

An example sotalol treatment protocol for a pediatric patient experiencing AFL, who is currently in normal sinus rhythm is described herein. The female patient, age 6 years, would be admitted to initiate treatment in a hospital or other facility capable of providing cardiac resuscitation and continuous electrocardiogramonitoring. The patient's creatinine clearance is determined to be 65 mL/min/1.73 m², which is indicative of mild renal impairment according to the age-specific creatine clearance ranges. The patient is connected to an electrocardiogram and an initial QTc is determined to be less than 450 ms. A physician determines the patient should be initiated on an IV sotalol dosing regimen. Treatment is initiated with an IV dose of sotalol hydrochloride in an amount of about 0.93 mg/kg over a period of about 5 hours. The patient's QTc is measured during or after the IV dose administration, along with heart rate and blood pressure. The patient's QTc does not exceed 500 ms during any of these measurements, and the ΔQTc is less than 20%.

Once it is determined that the patient/subject is capable of tolerating the sotalol by way of the IV dose, a subsequent IV or oral dose in an amount of about 0.93 mg/kg can be administered to the subject about 8 hours after initiation of the IV dose. The patient's QTc is monitored following administration of the subsequent IV or oral dose. The patient's QTc still does not exceed 500 ms and the patient's ΔQTc is less than 20%. Additional subsequent IV and/or oral doses in an amount of about 0.93 mg/kg would be administered to the patient every 8 hours.

### EXAMPLE 7-12 Year Old With Severe Renal Impairment

An example sotalol treatment protocol for a pediatric patient experiencing AF, who is currently in normal sinus rhythm is described herein. The female patient, age 12 years, would be admitted to initiate treatment in a hospital or other facility capable of providing cardiac resuscitation and continuous electrocardiogramonitoring. The patient's creatinine clearance is determined to be 12 mL/min/1.73 m², which indicates severe renal impairment according to the age-specific creatinine clearance ranges. The patient is connected to an electrocardiogramd an initial QTc is determined to be less than 450 ms. A physician determines the patient should be initiated on an IV sotalol dosing regimen. Treatment is initiated with an IV dose of sotalol hydrochloride in an amount of about 0.2-0.5 mg/kg (such as 0.43 mg/kg) over a period of about 1 hour. The patient's QTc is measured every 15 minutes during the IV dose administration, along with heart rate and blood pressure. The patient's QTc does not exceed 500 ms, however, the ΔQTc is more than 20%.

Once it is determined that the patient/subject is not capable of tolerating the sotalol by way of the IV dose, a subsequent IV dose in the range of 0.1-0.25 mg/kg (for example, an amount that is half of the initial IV dose amount, such as 0.22 mg/kg) can be administered over a period of 1 hour to the subject about 8 hours after initiation of the IV administration. The patient's QTc is monitored following administration of the subsequent IV dose. The patient's QTc still does not exceed 500 ms and the patient's ΔQTc is now less than 20%. Additional subsequent IV doses in an amount in the range of 0.1-0.2 mg/kg over a period of 1-5 hours would be administered to the patient every 8 hours.

The present disclosure has described particular implementations having various features. In light of the disclosure provided herein, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A method of administering sotalol hydrochloride treatment, comprising:
determining age and creatinine clearance of a pediatric patient;
selecting an amount of a single dose of sotalol hydrochloride to administer, wherein the amount of the single dose is an age-specific creatinine clearance based amount; and
administering the single dose of sotalol hydrochloride to the pediatric patient for treatment of a cardiovascular condition;
optionally administering one or more subsequent dose(s) of sotalol hydrochloride to the pediatric patient, which subsequent dose(s) comprise an amount of sotalol hydrochloride that is the same as or different from, or commensurate in effect with, the single dose.

2. The method of claim 1, wherein:
one or more of the single dose and/or the one or more subsequent dose(s) is in an amount in the range of:
(i) about 1.4-1.5 mg/kg, and the creatinine clearance of the patient is normal, and the age of the pediatric patient is in the range of 3 months to <6 months; or
(ii) about 1.35-1.45 mg/kg, and the creatinine clearance of the patient is normal, and the age of the pediatric patient is in the range of 6 months to <12 months; or
(iv) about 1.3-1.4 mg/kg, the creatinine clearance of the patient is normal, and the age of the pediatric patient is in the range of 12 months to <2 years.

3. The method of claim 2, wherein:
one or more of the single dose and/or the one or more subsequent dose(s) is in an amount in the range of about 1.4-1.5 mg/kg;
the creatinine clearance of the pediatric patient is greater than 53 mL/min/1.73m²; and
the age of the pediatric patient is 3 months to <6 months.

4. The method of claim 3, wherein:
one or more of the single dose and/or the one or more subsequent dose(s) is in an amount of 1.44 mg/kg.

5. The method of claim 2, wherein:
one or more of the single dose and/or the one or more subsequent dose(s) is in an amount in the range of about 1.35-1.45 mg/kg;
the creatinine clearance of the pediatric patient is greater than 68 mL/min/1.73m²; and
the age of the pediatric patient is 6 months to <12 months.

6. The method of claim 5, wherein:
one or more of the single dose and/or the one or more subsequent dose(s) is in an amount of 1.41 mg/kg.

7. The method of claim 2, wherein:
one or more of the single dose and/or the one or more subsequent dose(s) is in an amount in the range of about 1.3-1.4 mg/kg;
the creatinine clearance of the pediatric patient is greater than 81 mL/min/1.73m²; and
the age of the pediatric patient is 12 months to <2 years.

8. The method of claim 7, wherein:
one or more of the single dose and/or the one or more subsequent dose(s) is in an amount of 1.38 mg/kg.

9. The method of claim 1, wherein:
the selecting comprises following dosing instructions requiring administration of the single doses in an amount in the range of:
(i) 0.23-0.29 mg/kg, wherein the creatinine clearance of the pediatric patient is in the range of about 22 to 33 mL/min/1.73m² and the age of the pediatric patient is 5 days or less;
(ii) 0.42-0.47 mg/kg, wherein the creatinine clearance of the pediatric patient is in the range of about 22 to 34 mL/min/1.73m² and the age of the pediatric patient is 6 to <9 days;
(iii) 0.54-0.59 mg/kg, wherein the creatinine clearance of the pediatric patient is in the range of about 23-34 mL/min/1.73m² and the age of the pediatric patient is 9 to <12 days;
(iv) 0.60-0.73 mg/kg, wherein the creatinine clearance of the pediatric patient is in the range of about 23-35 mL/min/1.73m² and the age of the pediatric patient is 12 days to <2 weeks;
(v) 0.6-0.7 mg/kg, wherein the creatinine clearance of the pediatric patient is in the range of about 24 to 36 mL/min/1.73m² and the age of the pediatric patient is 2 weeks to <3 weeks;
(vi) 0.7-0.9 mg/kg, wherein the creatinine clearance of the pediatric patient is in the range of about 25 to 37 mL/min/1.73m² and the age of the pediatric patient is 3 weeks to <1 month;
(vii) 0.80-1.06 mg/kg, wherein the creatinine clearance of the pediatric patient is in the range of about 26 to 40 mL/min/1.73m² and the age of the pediatric patient is 1 month to <3 months;
(viii) 1.08-1.12 mg/kg, wherein the creatinine clearance of the pediatric patient is in the range of about 35 to 53 mL/min/1.73m² and the age of the pediatric patient is 3 months to <6 months;
(ix) 1.06-1.18 mg/kg, wherein the creatinine clearance of the pediatric patient is in the range of about 45 to 68 mL/min/1.73m² and the age of the pediatric patient is 6 months to <12 months;
(x) 1.04-1.14 mg/kg, wherein the creatinine clearance of the pediatric patient is in the range of about 54 to 81 mL/min/1.73m² and the age of the pediatric patient is 12 months to <2 years;
(xi) 0.92-1.12 mg/kg, wherein the creatinine clearance of the pediatric patient is in the range of about 60 to 90 mL/min/1.73m² and the age of the pediatric patient is 2 years to <6 years;
(xii) 0.8-1.0 mg/kg, wherein the creatinine clearance of the pediatric patient is in the range of about 60 to 90 mL/min/1.73m² and the age of the pediatric patient is 6-12 years; and
(xiii) 0.65-0.79 mg/kg, wherein the creatinine clearance of the pediatric patient is in the range of about 60 to 90 mL/min/1.73m² and the age of the pediatric patient is >12 years.

10. The method of claim 1, wherein:
a) the age of the pediatric patient is about 5 days or less, and one or more of the single dose and/or the one or more subsequent dose(s) is in an amount in the range of:
(i) about 0.15-0.2 mg/kg and wherein the creatinine clearance of the pediatric patient is about 11 to 22 mL/min/1.73m²; or
(ii) about 0.05-0.15 mg/kg and wherein the creatinine clearance of the pediatric patient is about 4 to 11 mL/min/1.73m²; or
b) the age of the pediatric patient is between 6 days and <9 days, and one or more of the single dose and/or the one or more subsequent dose(s) is in an amount ranging from:
(i) about 0.3-0.4 mg/kg and wherein the creatinine clearance of the pediatric patient is about 11 to 22 mL/min/1.73m²; or
(ii) about 0.1-0.3 mg/kg and wherein the creatinine clearance of the pediatric patient is about 4 to 11 mL/min/1.73m²; or
c) the age of the pediatric patient is in the range of about 9 days and <12 days, and one or more of the single dose and/or the one or more subsequent dose(s) is in an amount ranging from:
(i) about 0.3-0.5 mg/kg and wherein the creatinine clearance of the pediatric patient is about 11 to 23 mL/min/1.73m²; or
(ii) about 0.2 to about 0.3 mg/kg and wherein the creatinine clearance of the pediatric patient is about 4 to 11 mL/min/1.73m²; or
d) the age of the pediatric patient is in the range of about 12 days and <14 days, and one or more of the single dose and/or the one or more subsequent dose(s) is in an amount ranging from:
(i) about 0.4-0.5 mg/kg and wherein the creatinine clearance of the pediatric patient is about 12 to 23mL/min/1.73m²; or
(ii) about 0.2-0.4 mg/kg and wherein the creatinine clearance of the pediatric patient is about 4-12 mL/min/1.73m²; or
e) the age of the pediatric patient is in the range of 2 weeks to <3 weeks, and one or more of the single dose and/or the one or more subsequent dose(s) is in an amount ranging from:
(i) about 0.4-0.6 mg/kg and wherein the creatinine clearance of the pediatric patient is about 12 to 24 mL/min/1.73m²; or
(ii) about 0.3-0.4 mg/kg and wherein the creatinine clearance of the pediatric patient is about 4 to 12 mL/min/1.73m²; or
f) the age of the pediatric patient is in the range of 3 weeks to <1 month, and one or more of the single dose and/or the one or more subsequent dose(s) is in an amount ranging from:
(i) about 0.5-0.7 mg/kg and wherein the creatinine clearance of the pediatric patient is about 12 to 25 mL/min/1.73m²; or
(ii) about 0.3-0.5 mg/kg and wherein the creatinine clearance of the pediatric patient is about 4 to 12 mL/min/1.73m²; or
g) the age of the pediatric patient is in the range of 1 month to <3 months, and one or more of the single dose and/or the one or more subsequent dose(s) is in an amount ranging from:
(i) about 0.5-0.8 mg/kg and wherein the creatinine clearance of the pediatric patient is about 13 to 26 mL/min/1.73m²; or
(ii) about 0.3-0.5 mg/kg and wherein the creatinine clearance of the pediatric patient is about 4 to 13 mL/min/1.73m²; or
h) the age of the pediatric patient is in the range of about 2 to <6 years, and one or more of the single dose and/or the one or more subsequent dose(s) is in an amount ranging from:
(i) about 0.6-0.9 mg/kg and wherein the creatinine clearance of the pediatric patient is about 30 to 60 mL/min/1.73m²; or
(ii) about 0.3-0.6 mg/kg and wherein the creatinine clearance of the pediatric patient is about 10 to 30 mL/min/1.73m²; or
i) the age of the pediatric patient is in the range of about 6 to 12 years, and one or more of the single dose and/or the one or more subsequent dose(s) is in an amount ranging from:
(i) about 0.5-0.7 mg/kg and wherein the creatinine clearance of the pediatric patient is about 30 to 60 mL/min/1.73m²; or
(ii) about 0.2-0.5 mg/kg and wherein the creatinine clearance of the pediatric patient is about 10 to 30 mL/min/1.73m²; or
j) the age of the pediatric patient is greater than 12 years, and one or more of the single dose and/or the one or more subsequent dose(s) is in an amount ranging from:
(i) about 0.4-0.6 mg/kg and wherein the creatinine clearance of the pediatric patient is about 30 to 60 mL/min/1.73m²; or
(ii) about 0.2-0.4 mg/kg and wherein the creatinine clearance of the pediatric patient is about 10 to 30 mL/min/1.73m².

11. The method of claim 1, comprising:
administering the single dose of sotalol hydrochloride to the pediatric patient in an amount of 1.21 mg/kg at the age of 1 month, wherein the creatinine clearance of the pediatric patient is >40 mL/min/1.73m²; and
administering one or more of the subsequent doses to the pediatric patient in an amount of 1.44 mg/kg at the age of 3 months, wherein the creatinine clearance of the pediatric patient is >53 mL/min/1.73m².

12. The method of claim 1, wherein the selecting comprises reviewing dosing instructions, wherein the dosing instructions include instructions to administer a higher amount of sotalol hydrochloride to a pediatric patient who is 2 years old, than to a pediatric patient who is 1 month old.

13. The method of claim 1, comprising:
wherein the selecting comprises reviewing dosing instructions, wherein the dosing instructions include instructions to administer a higher amount of sotalol hydrochloride to a pediatric patient who is 3 months old than to a pediatric patient who is 1 month old.

14. The method of claim 1, comprising:
wherein the selecting comprises referring to dosing instructions requiring administration of 1.38 mg/kg of sotalol hydrochloride to a pediatric patient that is 12 months old and 1.21 mg/kg to a pediatric patient who is 1 month old.

15. The method of claim 1, comprising:
determining the pediatric patient is 3 months old;
determining the pediatric patient has a normal creatinine clearance; and
administering a higher amount of sotalol hydrochloride to the pediatric patient at 3 months old than would be given the pediatric patient at 6 years old.

16. The method of claim 1, comprising:
determining the pediatric patient is 3 months old and administering a single dose and one or more of the subsequent doses of sotalol hydrochloride in an amount of 1.44 mg/kg to the pediatric patient; and
administering at least one dose of sotalol hydrochloride in an amount of 1.38 mg/kg to a second pediatric patient who is 12 months old.

17. The method of claim 1, wherein:
in response to administering the single dose, the pediatric patient is capable of achieving a peak serum, plasma or blood concentration of sotalol hydrochloride in the range of 90-110% of a Cₘₐₓ at steady-state that would be expected for the pediatric patient from oral dosing.

18. The method of claim 1, wherein:
the pediatric patient is currently in sinus rhythm, or is not currently in sinus rhythm, or has been or will be converted to sinus rhythm with sotalol or another antiarrhythmic and/or cardioversion such as electric cardioversion; and
the pediatric patient has a cardiovascular condition selected from one or more of atrial flutter (AFL), atrial fibrillation (AF), persistent atrial fibrillation, paroxysmal atrial fibrillation, paroxysmal or persistent atrial fibrillation or atrial flutter, a recent AF/AFL episode, ventricular fibrillation (VF) or ventricular arrhythmias, life-threatening recurrent VF or life-threatening recurrent hemodynamically unstable VT, focal atrial tachycardia, supraventricular tachycardia (SVT), atrioventricular re-entrant tachycardia (AVRT), atrioventricular nodal re-entrant tachycardia (AVNRT), atrial tachycardia, paroxysmal atrial tachycardia, junctional tachycardia, junctional ectopic tachycardia (JET), congenital heart disease (CHD), multifocal atrial tachycardia, atrial ectopic tachycardia, accessory pathway SVT, infantile SVT, post-operative SVT, paroxysmal SVT, treating Wolff-Parkinson-White syndrome, ventricular tachycardia (VT), premature ventricular contractions (PVCs), hemodynamically stable or unstable ventricular tachycardia, heart failure, coronary artery disease, pulmonary artery hypertension, and/or life- threatening ventricular tachycardia.

19. The method of claim 1, wherein:
the single dose is an IV dose; and
one or more of the subsequent dose(s) are administered as IV dose(s).

20. The method of claim 1, wherein:
the single dose and/or the one of more subsequent IV doses are administered as a bolus dose, or over a period of up to 1 hour, or over a period of about 30 min., or over a period of 1 hour, or over a period of up to 5 hours, or over a period of 5 hours.
